(19) United States
(12) Patent Application Publication
(10) Pub. No.: US 2013/0090275 A1
(43) Pub. Date: Apr. 11, 2013

(54) LUBRICATING OIL COMPOSITION FOR FLUID DYNAMIC BEARINGS AND HDD MOTOR FABRICATED USING THE SAME

(71) Applicants: Myung Hwa CHOI, Suwon (KR); Sang Hyun Kwon, Suwon (KR); Hyung Kyu Kim, Daejeon (KR); Ha Yong Jung, Suwon (KR)

(72) Inventors: Myung Hwa CHOI, Suwon (KR); Sang Hyun Kwon, Suwon (KR); Hyung Kyu Kim, Daejeon (KR); Ha Yong Jung, Suwon (KR)

(73) Assignee: SAMSUNG ELECTRO-MECHANICS CO., LTD.

(21) Appl. No.: 13/331,466

(22) Filed: Dec. 20, 2011

(30) Foreign Application Priority Data
Oct. 10, 2011 (KR) 10-2011-0102953

(51) Int. Cl. C10M 135/10; C10M 137/04; C07C 69/02; C10M 129/08

(52) U.S. Cl. 508/404; 508/505; 508/440; 560/129

(57) ABSTRACT

There are provided a lubricating oil composition for a fluid dynamic bearing and and a hard disk drive (HDD) motor fabricated by using the same. The lubricating oil composition for a fluid dynamic bearing includes: an aliphatic mono carboxylic acid ester, as a base oil, obtained by esterification between carboxylic acid represented by the following Chemical Formula 1; and isopropyl alcohol:

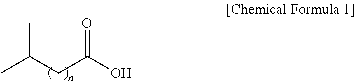

where n indicates an integer of 6 to 20. Therefore, the HDD motor is fabricated by using the lubricating oil composition for a fluid dynamic bearing having low viscosity, low evaporation loss, and improved oxidation stability at room temperature, whereby quality reliability according to the use of the motor for a long period of time may be improved.

### CROSS-REFERENCE TO RELATED APPLICATIONS

This application claims the priority of Korean Patent Application No. 10-2011-0102953 filed on Oct. 10, 2011, in the Korean Intellectual Property Office, the disclosure of which is incorporated herein by reference.

### BACKGROUND OF THE INVENTION

1. Field of the Invention

The present invention relates to a lubricating oil composition for a fluid dynamic bearing having low viscosity, low evaporation loss, and improved oxidation stability, and a hard disk drive (HDD) motor fabricated by using the same.

2. Description of the Related Art

A hard disk drive (HDD), an information storage device, reads data stored on a disk or writes data to the disk using a read/write head.

The hard disk drive requires a disk driving device capable of driving a disk. In the disk driving device, a small-sized spindle motor is used.

The small-sized spindle motor has used a fluid dynamic bearing assembly. A lubricating fluid is interposed between a shaft and a sleeve of the fluid dynamic bearing assembly, such that the shaft is supported by fluid pressure generated in the lubricating fluid.

When the lubricating fluid may have high viscosity at a low temperature at the time of rotation of the spindle motor, viscous resistance of the lubricating fluid to a groove generating dynamic force at the time of the rotation of the motor, increases, thereby increasing power loss in the motor.

On the other hand, the lubricating fluid may be thermally expanded and have a reduced viscosity at a high temperature at the time of the rotation of the spindle motor, such that it may not sufficiently perform a support role.

Due to the above-mentioned defect, the lubricating fluid requires reversed viscosity behavior characteristics, in which low viscosity is maintained in a low temperature region, while viscosity is not reduced in a high temperature region.

In order to satisfy these viscosity characteristics, several methods, such as a method of adding a material such as an anti-oxidant, an pressure preventing additive, or the like, to a base oil including a specific ester compound as a main component, have been developed.

The lubricating fluid to which the above-mentioned additives are added may demonstrate initial viscosity effects. However, when a small sized spindle motor is used for an extended period of time, the lubricant may be evaporated and viscous characteristics thereof may be changed, such that it may be difficult to continuously maintain this effect.

In addition, in accordance with the trend for miniaturization, high precision, high speed rotation, and low power consumption in the motor, characteristics such as heat resistance, oxidation stability, low evaporation, and abrasion prevention have been demanded in the lubricating fluid.

Meanwhile, when a viscosity of the base oil is reduced, evaporation loss tends to increase. Therefore, a base oil having low viscosity at room temperature able to suppress evaporation loss has been demanded.

### SUMMARY OF THE INVENTION

An aspect of the present invention provides a lubricating oil composition for a fluid dynamic bearing having low viscosity, low evaporation loss, and improved oxidation stability, and a hard disk drive (HDD) motor fabricated by using the same.

According to an aspect of the present invention, there is provided a lubricating oil composition for a fluid dynamic bearing including: an aliphatic mono carboxylic acid ester, as a base oil, obtained by esterification between carboxylic acid represented by the following Chemical Formula 1; and isopropyl alcohol:

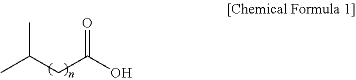

where n indicates an integer of 6 to 20.

The aliphatic mono carboxylic acid ester may be represented by the following Chemical Formula 3:

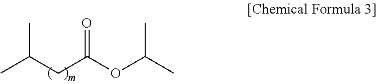

where m indicates an integer of 6 to 20.

The aliphatic mono carboxylic acid ester may be at least one selected from a group consisting of isopropyl isopalmitate and isopropyl isostearate.

The lubricating oil composition may further include 0.01 to 2 parts by weight of an oil antioxidant, wherein the oil antioxidant is 2,2′-methylene-bis(4-methyl-6-tert-butylphenol).

The lubricating oil composition may further include 0.01 to 2 parts by weight of a metal antioxidant, wherein the metal antioxidant is barium diphenylamine-4-sulfonate.

The lubricating oil composition may further include 0.01 to 2 parts by weight of an internal pressure preventing agent, wherein the internal pressure preventing agent is tricresyl phosphate.

According to another aspect of the present invention, there is provided a hard disk drive (HDD) motor including: a lubricating oil composition for a fluid dynamic bearing including an aliphatic mono carboxylic acid ester, as a base oil, obtained by esterification between carboxylic acid represented by the following Chemical Formula 1; and isopropyl alcohol:

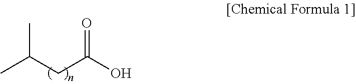

where n indicates an integer of 6 to 20.

The aliphatic mono carboxylic acid ester may be represented by the following Chemical Formula 3:

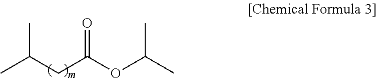

where m indicates an integer of 6 to 20.

The aliphatic mono carboxylic acid ester may be at least one selected from a group consisting of isopropyl isopalmitate and isopropyl isostearate.

The lubricating oil composition may further include 0.01 to 2 parts by weight of an oil antioxidant, wherein the oil antioxidant is 2,2′-methylene-bis(4-methyl-6-tert-butylphenol).

The lubricating oil composition may further include 0.01 to 2 parts by weight of a metal antioxidant, wherein the metal antioxidant is barium diphenylamine-4-sulfonate.

The lubricating oil composition may further include 0.01 to 2 parts by weight of an internal pressure preventing agent, wherein the internal pressure preventing agent is tricresyl phosphate.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a cross-sectional view schematically showing a hard disk drive (HDD) motor including a fluid dynamic bearing assembly according to an embodiment of the present invention.

### DETAILED DESCRIPTION OF THE INVENTION

Embodiments of the present invention may be modified in many different forms and the scope of the invention should not be seen as being limited to the embodiments set forth herein. Rather, these embodiments are provided so that this disclosure will be thorough and complete, and will fully convey the concept of the invention to those skilled in the art. In the drawings, the shapes and dimensions of elements may be exaggerated for clarity, and the same reference numerals will be used throughout to designate the same or like components.

Embodiments of the present invention will now be described in detail with reference to the accompanying drawings.

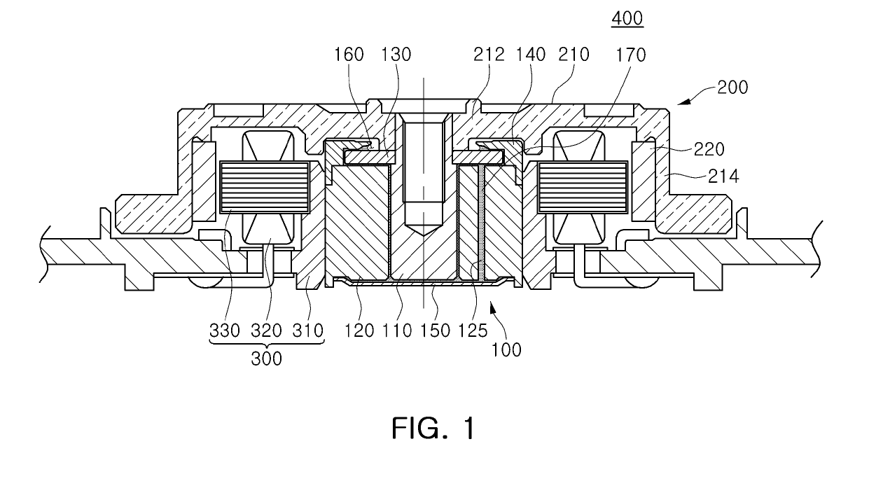

FIG. 1 is a cross-sectional view schematically showing a hard disk drive (HDD) motor including a fluid dynamic bearing assembly according to an embodiment of the present invention.

Referring to FIG. 1, a lubricating oil composition 170 for a fluid dynamic bearing according to the embodiment of the present invention may include aliphatic mono carboxylic acid ester, as a base oil, obtained by esterification between carboxylic acid represented by the following Chemical Formula 1; and isopropyl alcohol:

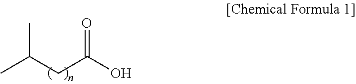

where n indicates an integer of 6 to 20.

Hereinafter, the above configuration will be described in detail.

The carboxylic acid represented by the above Chemical Formula 1 is not particularly limited as long as it is iso acid, and n may be an integer of 6 to 20.

As a specific example, the carboxylic acid may be iso-nonanoic acid, iso-decanoic acid, iso-undecanoic acid, iso-dodecanoic acid, iso-tridecanoic acid, iso-tetradecanoic acid, iso-pentadecanoic acid, iso-hexadecanoic acid, iso-heptadecanoic acid, iso-octadecanoic acid, iso-nonadecanoic acid, iso-icosanoic acid, iso-henicosanoic acid, iso-docosanoic acid, and iso-tricosanoic acid, but is not limited thereto.

Meanwhile, according to the embodiment of the present invention, in order to perform esterification with the carboxylic acid represented by the above Chemical Formula 1, isopropyl alcohol may be used as alcohol.

A structure of the isopropyl alcohol maybe represented by the following Chemical Formula 2.

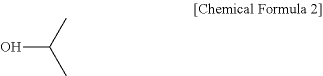

The lubricating oil composition 170 for a fluid dynamic bearing according to the embodiment of the present invention may include the aliphatic mono carboxylic acid ester, as a base oil, obtained by esterification between the carboxylic acid represented by the above Chemical Formula 1; and the isopropyl alcohol represented by the above Chemical Formula 2.

The aliphatic mono carboxylic acid ester may have, for example, a total of 12 to 26 carbon numbers, but is not limited thereto.

The aliphatic mono carboxylic acid ester may be represented by the following Chemical Formula 3:

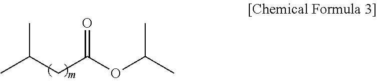

where m indicates an integer of 6 to 20.

The aliphatic mono carboxylic acid ester represented by the above Chemical Formula 3 is not particularly limited, and m may be an integer of 6 to 20.

More specifically, the aliphatic mono carboxylic acid ester may be at least one selected from a group consisting of isopropyl isopalmitate and isopropyl isostearate, but is not limited thereto.

The isopropyl isopalmitate and the isopropyl isostearate may be represented by the following Chemical Formulas 4 and 5.

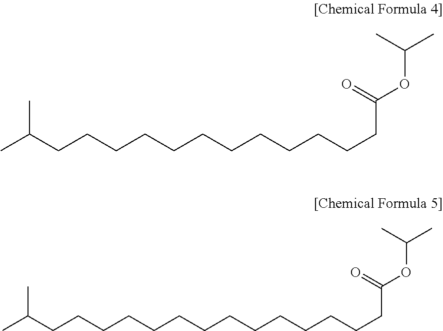

A kinematic viscosity of the aliphatic mono carboxylic acid ester according to the embodiment of the present invention may be measured at a temperature of −20° C., 25° C., and 85° C.

The viscosity may be measured using a Brookfield DB-III Rheometer Viscometer and be measured for each component at three temperature periods −20° C., 25° C., and 85° C. in order to confirm viscosity tendency according to temperature.

Among the three temperatures, −20° C. corresponds to a low temperature storage temperature, 25° C. corresponds to a room temperature operating temperature of a general motor, and 85° C. corresponds to a high temperature operating temperature of the motor, in a reliability test of the motor.

According to the embodiment of the present invention, the aliphatic mono carboxylic acid ester may have a viscosity and a high temperature evaporation amount that are lower than those of the aliphatic mono carboxylic acid ester obtained by esterification between dioctyl adipate (DOA) and dioctyl sebacate (DOS) or dioctyl azelate (DOZ).

Therefore, in the case in which the aliphatic mono carboxylic acid ester is used as the base oil, frictional loss in a device may be more effectively reduced while having a relatively low viscosity. In addition, an evaporation amount is relatively low, such that stability at high temperatures may be significantly excellent.

The lubricating oil composition including the aliphatic mono carboxylic acid ester as the base oil may be appropriate for being used as, for example, a fluid bearing of the HDD motor, but is not limited thereto.

In the case of a small-sized hard disk drive, a low power consumption amount needs to be relatively low, and stability at high temperatures may be very important due to high speed rotation of the motor.

The lubricating oil composition according to the embodiment of the present invention may have relatively low frictional loss and also have stability at high temperatures to thereby satisfy the above-mentioned conditions of the small-sized hard disk drive.

The lubricating oil composition for a fluid dynamic bearing may further include 0.01 to 2 parts by weight of an oil antioxidant. The oil antioxidant may be, for example, 2,2′-methylene-bis(4-methyl-6-tert-butylphenol), but is not limited thereto.

A content of the oil antioxidant may be 0.01 to 2 parts by weight in a range in which performance of the lubricating oil composition is not deteriorated. When the content of the oil antioxidant is less than 0.01 parts by weight, an effect of adding the antioxidant may be relatively small, and when the content of the oil antioxidant exceeds 2 parts by weight, the performance of the lubricating oil composition may be deteriorated.

In addition, the lubricating oil composition for a fluid dynamic bearing may further include 0.01 to 2 parts by weight of a metal antioxidant. The metal antioxidant may be, for example, barium diphenylamine-4-sulfonate, but is not particularly limited thereto.

When a content of the metal antioxidant is less than 0.01 parts by weight, the effect of stabilizing oxidation may be small, and when the content of the metal antioxidant exceeds 2 parts by weight, the performance of the lubricating oil composition may be deteriorated. Therefore, the content of the metal antioxidant may be in a range of 0.01 to 2 parts by weight.

The lubricating oil composition for a fluid dynamic bearing may further include 0.01 to 2 parts by weight of an internal pressure preventing agent. The internal pressure preventing agent may be, for example, tricresyl phosphate, but is not limited thereto.

When a content of the internal pressure preventing agent is less than 0.01 parts by weight, the effect of preventing internal pressure may be relatively small, and when the content of the internal pressure preventing agent exceeds 2 parts by weight, the performance of the lubricating oil composition may be deteriorated. Therefore, the content of the internal pressure preventing agent may be in a range of 0.01 to 2 parts by weight.

A HDD motor according to another embodiment of the present invention may include a lubricating oil composition for a fluid dynamic bearing including aliphatic mono carboxylic acid ester having a total of 26 to 40 carbon numbers, as a base oil, obtained by esterification between alcohol represented by the following Chemical Formula 1; and straight or branched saturated aliphatic mono carboxylic acid having 2 to 16 carbon numbers:

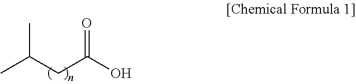

where n indicates an integer of 6 to 20.

Hereinafter, the HDD motor according to another embodiment of the present invention will be described in detail. However, a portion overlapped with the description in the above-mentioned embodiment of the present invention will be omitted.

The HDD motor 400 may include an oil sealing part 160 formed between fixed members 120 and 140 and rotating members 110, 130, and 212, particularly, between a sleeve 120, a thrust plate 130, and a cap 140.

The cap 140 may be a member that is press-fitted onto an upper portion of the thrust plate 130 to thereby allow a lubricating fluid to be sealed between the cap 140 and the thrust plate 130, and include a circumferential groove formed in a circumferential surface thereof so as to be press-fitted into the thrust plate 130 and the sleeve 120.

The cap 140 may include a protrusion part formed on a lower surface thereof in order to seal the lubricating fluid, which uses a capillary phenomenon and a surface tension of the lubricating fluid in order to prevent the lubricating fluid from being leaked to the outside at the time of driving of the motor.

Meanwhile, a HDD motor 400 according to another embodiment of the present invention may include a shaft 110, the sleeve 120, the thrust plate 130, the cap 140, and the oil sealing part 160.

The sleeve 120 may support the shaft 110 such that an upper end thereof protrudes upwardly in an axial direction, and maybe formed by forging Cu or Al or sintering Cu—Fe based alloy powders or SUS based powders.

Here, the shaft 110 may be inserted into a shaft hole of the sleeve 120 so as to have a micro clearance therewith. The micro clearance may be filled with the lubricating fluid, and the rotation of a rotor 200 may be more smoothly supported by a radial dynamic groove formed in at least one of an outer circumferential surface of of the shaft 110 and an inner circumferential surface of the sleeve 120.

The radial dynamic groove may be formed in an inner side of the sleeve 120, which is an inner portion of the shaft hole of the sleeve 120, and may generate pressure so as to be deflected toward one side at the time of rotation of the shaft 110.

However, the radial dynamic groove is not limited to being formed in the inner side of the sleeve 120 as described above but may also be formed in an outer circumferential surface portion of the shaft 110. In addition, the number of radial dynamic grooves is not limited.

The sleeve 120 may include a bypass channel 125 formed therein in order to allow upper and lower portions thereof to be in communication with each other to disperse pressure of the lubricating fluid in an inner portion of a fluid dynamic bearing assembly 100, thereby maintaining balance in the pressure, and may move air bubbles, or the like, present in the inner portion of the fluid dynamic bearing assembly 100, to be discharged by circulation.

Here, the sleeve 120 may include a cover plate 150 coupled to a lower portion thereof, having a clearance therebetween, wherein the clearance receives the lubricating fluid therein.

The cover plate 150 may receive the lubricating fluid in the clearance between the cover plate 150 and the sleeve 120 to thereby serve as a bearing supporting a lower surface of the shaft 110.

The thrust plate 130 may be disposed on an upper portion of the sleeve 120 in the axial direction and includes a hole formed at the center thereof, wherein the hole is formed to correspond to a cross section of the shaft 110. The shaft 110 may be inserted into this hole.

Here, the thrust plate 130 maybe separately fabricated and then coupled to the shaft 110. However, the thrust plate 130 may be formed integrally with the shaft 110 at the time of fabricating thereof and may rotate together with the shaft 110 at the time of the rotation of the shaft 110.

In addition, the thrust plate 130 may include a thrust dynamic groove formed in an upper surface thereof, wherein the thrust dynamic groove provides thrust dynamic pressure to the shaft 110.

The thrust dynamic groove is not limited to being formed in the upper surface of the thrust plate 130 as described above but may also be formed in an upper surface of the sleeve 120 corresponding to a lower surface of the thrust plate 130.

The stator 300 may include a coil 320, a core 330, and a base member 310.

In other words, the stator 300 may be a fixed structure including the coil 320 generating electromagnetic force having a predetermined magnitude at the time of application of power and a plurality of cores 330 having the coil 320 wound therearound.

The core 330 is fixedly disposed on an upper portion of a base member 310 on which a printed circuit board (not shown) having pattern circuits printed thereon is provided, a plurality of coil holes having a predetermined size are formed to penetrate through the base member so as to expose the winding coil 320 downwardly, penetrating a portion of the base member 310 corresponding to the winding coil 320, and the winding coil 320 may be electrically connected to the printed circuit board (not shown) in order to supply external power.

The base member 310 may be press-fitted and fixed onto an outer peripheral surface of the sleeve 120 and have the core 330 inserted into an inner portion thereof, wherein the core 330 has the coil 320 wound therearound.

In addition, the base member 310 and the sleeve 120 may be assembled to each other by applying an adhesive to an inner surface of the base member 310 or an outer surface of the sleeve 120.

The rotor 200, a rotational structure rotatably provided with respect to the stator 300, may include a rotor case 210 having an annular ring shaped magnet 220 provided on an outer peripheral surface thereof, wherein the annular ring shaped magnet 220 corresponds to the core 330, having a predetermined interval therebetween.

Here, as the magnet 220, a permanent magnet generating magnetic force having predetermined strength by alternately magnetizing an N pole and an S pole thereof in a circumferential direction may be used.

Here, the rotor case 210 may include a hub base 212 press-fitted into the upper end of the shaft 110 to thereby be fixed thereto and a magnet support part 214 extended from the hub base 212 in an outer diameter direction and bent downwardly in the axial direction to thereby support the magnet 220.

A HDD motor according to another embodiment of the present invention may be fabricated by using the lubricating oil composition 170 for a fluid dynamic bearing, whereby frictional loss in a device may be more effectively reduced while having a relatively low viscosity. In addition, an evaporation amount is low, such that stability at high temperatures may be significantly excellent.

In addition, the HDD motor may be fabricated by using the lubricating oil composition for a fluid dynamic bearing having a low viscosity, low evaporation loss, and improved oxidation stability at room temperature, whereby quality reliability according to the use of the motor for a long period of time may be improved.

A fabricating method of the HDD motor 400 may be the same as a general fabricating method except that the HDD motor 400 is fabricated by using the lubricating oil composition 170 for a fluid dynamic bearing.

Hereafter, although the present invention will be described in detail with reference to Comparative Example and Inventive Example, it is not limited thereto.

### INVENTIVE EXAMPLE

In the case of Inventive Example, isopropyl isostearate was synthesized by allowing isostearic acid and isopropyl alcohol available from the Sigma A. Co. to react with each other.

As conditions of the reaction, alcohol and acid were introduced in a reactor and then left at a temperature of 200° C. for 24 hours. After the reaction, a purifying process was performed.

The isopropyl isostearate occupied about 95 wt % based on the entire weight ratio, and remaining 5 wt % of additive was added thereto in order to improve other characteristics.

More specifically, 2 wt % of 2,2′-methylene-bis(4-methyl-6-tert-butylphenol) was added in order to prevent oxidation of oil, and 2 wt % of tricresyl phosphate was added as an internal pressure preventing agent.

In addition, 1 wt % of barium diphenylamine-4-sulfonate was added in order to prevent oxidation of a metal surface contacting the oil.

### COMPARATIVE EXAMPLES 1 and 2

In the case of Comparative Example 1, a lubricating oil composition was prepared by esterification between dioctyl sebacate (DOS) and dioctyl adipate (DOA), and in the case of Comparative Example 2, a lubricating oil composition was prepared by esterification between dioctyl azelate (DOZ) and dioctyl adipate (DOA). In both of Comparative Examples 1 and 2, a type and contents of other additives were the same as those in Inventive Example.

Esters of Comparative Examples 1 and 2 may be represented by the following Chemical Formulas 6 and 7:

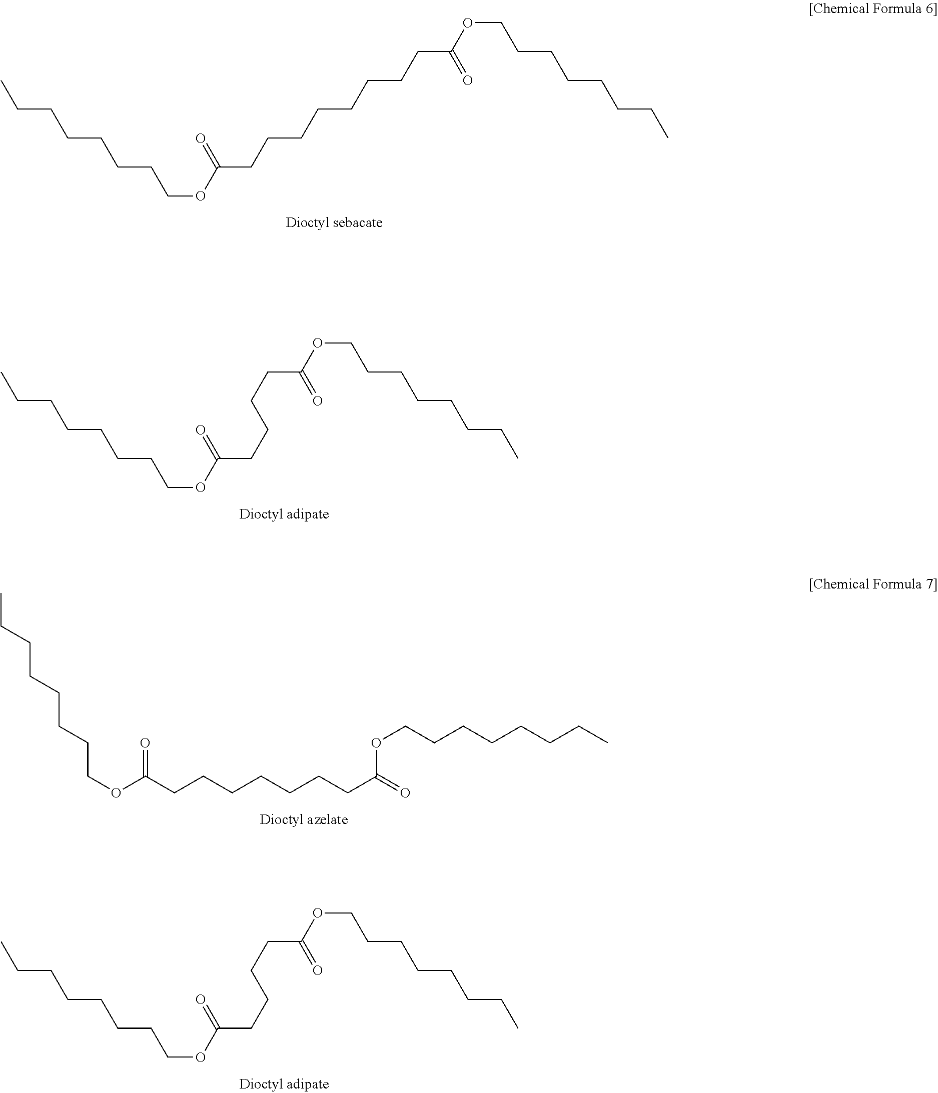

The following Table 1 shows that viscosities for comparing performances of lubricating oil compositions according to Inventive Example and Comparative Examples with each other and evaporation amounts for comparing reliabilities thereof with each other are measured and compared with each other.

The viscosities were measured using the Brookfield DB-III Rheometer viscometer and were measured for each component at three temperature periods of −20° C., 25° C., and 85° C. in order to confirm inclination according to a temperature.

An experiment of measuring the evaporation amounts was performed by putting each of 5 grams of lubricating oil compositions for a fluid dynamic bearing including each component on an evaporation dish formed of an SUS material and then introducing a thermostat of 100° C. to it.

The experiment was performed for 144 hours (six days), and an evaporation amount was compared with each other to compare evaporation amounts by measuring initial weight of the lubricating oil composition put on the evaporation dish and weight of the lubricating oil composition after 144 hours elapse in the thermostat of 100° C.

TABLE 1

| | Viscosity (cP) | | | Evaporation Amount (wt %) |
|---|---|---|---|---|
| Division | −20° C. | 25° C. | 85° C. | (100° C., 144 h) |
| Inventive | 138.4 | 12.1 | 3.00 | 5.8 |
| Example | | | | |
| Comparative | 167.9 | 14.0 | 3.49 | 6.8 |
| Example 1 | | | | |
| Comparative | 160.4 | 13.4 | 3.30 | 7.1 |
| Example 2 | | | | |

It can be appreciated from the above Table 1 that the lubricating oil composition according to the present invention (Inventive Example) has a viscosity and an evaporation amount lower than those of the lubricating oil composition by the esterification between the dioctyl sebacate (DOS) and the dioctyl adipate (DOA) (Comparative Example 1) and the lubricating oil composition by the esterification between the dioctyl azelate (DOZ) and the dioctyl adipate (DOA) (Comparative Example 2).

As set forth above, according to the embodiments of the present invention, the HDD motor may be fabricated by using the lubricating oil composition for a fluid dynamic bearing having a relatively low viscosity, low evaporation loss, and improved oxidation stability at room temperature, whereby quality reliability according to the use of the motor for a long period of time may be improved.

While the present invention has been shown and described in connection with the embodiments, it will be apparent to those skilled in the art that modifications and variations can be made without departing from the spirit and scope of the invention as defined by the appended claims.

What is claimed is:

1. A lubricating oil composition for a fluid dynamic bearing, comprising:
an aliphatic mono carboxylic acid ester, as a base oil, obtained by esterification between carboxylic acid represented by the following Chemical Formula 1; and isopropyl alcohol: 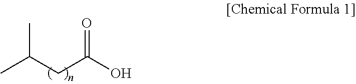
where n indicates an integer of 6 to 20.

2. The lubricating oil composition of claim 1, wherein the aliphatic mono carboxylic acid ester is represented by the following Chemical Formula 3: 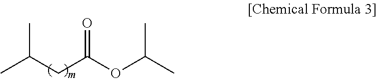
where m indicates an integer of 6 to 20.

3. The lubricating oil composition of claim 1, wherein the aliphatic mono carboxylic acid ester is at least one selected from a group consisting of isopropyl isopalmitate and isopropyl isostearate.

4. The lubricating oil composition of claim 1, further comprising 0.01 to 2 parts by weight of an oil antioxidant.

5. The lubricating oil composition of claim 4, wherein the oil antioxidant is 2,2′-methylene-bis(4-methyl-6-tert-butylphenol).

6. The lubricating oil composition of claim 1, further comprising 0.01 to 2 parts by weight of a metal antioxidant.

7. The lubricating oil composition of claim 6, wherein the metal antioxidant is barium diphenylamine-4-sulfonate.

8. The lubricating oil composition of claim 1, further comprising 0.01 to 2 parts by weight of an internal pressure preventing agent.

9. The lubricating oil composition of claim 8, wherein the internal pressure preventing agent is tricresyl phosphate.

10. A hard disk drive (HDD) motor comprising:
a lubricating oil composition for a fluid dynamic bearing including an aliphatic mono carboxylic acid ester, as a base oil, obtained by esterification between carboxylic acid represented by the following Chemical Formula 1; and isopropyl alcohol: 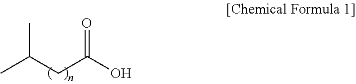
where n indicates an integer of 6 to 20.

11. The HDD motor of claim 10, wherein the aliphatic mono carboxylic acid ester is represented by the following Chemical Formula 3: 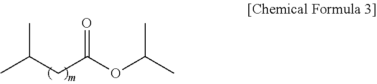
where m indicates an integer of 6 to 20.

12. The HDD motor of claim 10, wherein the aliphatic mono carboxylic acid ester is at least one selected from a group consisting of isopropyl isopalmitate and isopropyl isostearate.

13. The HDD motor of claim 10, wherein the lubricating oil composition further includes 0.01 to 2 parts by weight of an oil antioxidant.

14. The HDD motor of claim 13, wherein the oil antioxidant is 2,2′-methylene-bis(4-methyl-6-tert-butylphenol).

15. The HDD motor of claim 10, wherein the lubricating oil composition further includes 0.01 to 2 parts by weight of a metal antioxidant.

16. The HDD motor of claim 15, wherein the metal antioxidant is barium diphenylamine-4-sulfonate.

17. The HDD motor of claim 10, wherein the lubricating oil composition further includes 0.01 to 2 parts by weight of an internal pressure preventing agent.

18. The HDD motor of claim 17, wherein the internal pressure preventing agent is tricresyl phosphate.
